(19) United States
(12) Patent Application Publication
(10) Pub. No.: US 2013/0344104 A1
(43) Pub. Date: Dec. 26, 2013

(54) OLIGOSACCHARIDES AND OLIGOSACCHARIDE-PROTEIN CONJUGATES DERIVED FROM CLOSTRIDIUM DIFFICILE POLYSACCHARIDE PS-II, METHODS OF SYNTHESIS AND USES THEREOF, IN PARTICULAR AS A VACCINE

(71) Applicants: Peter H. Seeberger, Kleinmachnow (DE); Matthias Oberli, Cambridge, MA (US); Marie-Lyn Hecht, Buchrain (CH)

(72) Inventors: Peter H. Seeberger, Kleinmachnow (DE); Matthias Oberli, Cambridge, MA (US); Marie-Lyn Hecht, Buchrain (CH)

(21) Appl. No.: 14/003,801

(22) PCT Filed: Mar. 7, 2012
(86) PCT No.: PCT/EP12/01014

§ 371 Date: Sep. 8, 2013

Related U.S. Application Data
(60) Provisional application No. 61/450,388, filed on Mar. 8, 2011.

(51) Int. Cl. A61K 39/08; C08B 37/00; C07K 16/12; C07K 14/34

(52) U.S. Cl. CPC A61K 39/08; C07K 14/34; C08B 37/0009; C07K 16/1282
USPC 424/197.11; 530/395; 536/123.1; 424/247.1; 530/387.5; 530/363

(57) ABSTRACT

The present invention provides an oligosaccharide-protein conjugate comprising an oligosaccharide, in particular synthetic oligosaccharide, derived from the repeating unit of the Clostridium difficile glycopolymer PS-II and a protein carrier. More specifically, the oligosaccharide is the hexasaccharide having the following formula (I) wherein R is a linker or spacer group. In a specific embodiment of the invention, R is (CH2)ₙNH₂, with n being an integer from 2 to 50. The present invention also provides the use of said oligosaccharide and said oligosaccharide-protein conjugate for the treatment or prevention of a disease caused by the pathogen Clostridium difficile. In still further aspects, the present invention also provides a favourable method for preparing said oligosaccharide and said oligosaccharide-protein conjugate.

### BACKGROUND

The Gram-positive bacteria of the genus Clostridium difficile have long been recognized as the cause of a range of gastrointestinal diseases. Infection and the development of Clostridium difficile associated diseases (CDAD) are linked to the use of antibiotics that disrupt the normal intestinal flora and allow for proliferation of C. difficile. C. difficile infections in its most severe form can cause toxic megacolon with subsequent colonic perforation, peritonitis, shock, and death. Furthermore, C. difficile is a major cause of diarrhea in hospital- and long-term care facility patients due to the frequent use of antibiotics, contamination of these facilities with resistant spores and because of the high density of susceptible persons. A dramatic increase in C. difficile incidents was recorded in many developed countries, starting with reports of hospital outbreaks in Canada in 2003. With increasing severity of the incidents, relapse and mortality rates also increased significantly. The North American and European outbreaks coincided with the emergence of a hypervirulent strain of C. difficile, alternatively designated by the synonymous terms as PCR ribotype 027, toxintype III, NAP1, and BI (McDonald et al. (2005), N. Engl. J. Med. 353, 2433-2441; Loo et al. (2005), N. Engl. J. Med. 353, 2442-2449). The hypervirulence of ribotype 027 has been ascribed to its higher toxin yields and an increased rate of sporulation. Higher toxin content is due to an additional toxin referred to as the binary toxin and a genetic mutation in a toxin regulator gene (tcdC), encoding a negative regulator of the C. difficile pathogenicity locus. The isolates obtained during the North American and European epidemics were genetically closely related and in addition resistant to fluoroquinolones.

In conclusion, the pathogen C. difficile represents a major risk for patients and causes significant costs to health care systems. Unfortunately, however, currently no licensed vaccine against C. difficile is available.

Thus, a main object of the present invention is to provide novel and effective means to prevent and/or treat C. difficile associated diseases, in particular related to the hypervirulent strain ribotype 027.

Preventive vaccination with bacterial cell-surface polysaccharides, either isolated from natural sources or synthetically produced, has shown to be an effective measure against some bacteria such as Haemophilus influenza type b, Streptococcus pneumoniae, Neisseria meningitidis, Salmonella typhi, and Staphylococcus aureus (Ada et al. (2003), Clin. Microbiol. Infect. 9, 79-85). A known approach in vaccine development is the application of natural polysaccharides either neat (Lucas et al. (1999), Immunol. Rev. 171, 89-1049) or linked to immunogenic protein carriers (Hecht et al., Curr. Opin. Chem. Biol. 13, 354-359. (2009)).

Studies aiming at elucidating the structural composition of the C. difficile cell wall resulted in the identification of two capsular polysaccharides PS-I and PS-II (Ganeshapillai et al. (2008), Carbohydr. Res. 343, 703-710); US 20100330125). PS-I is composed of a branched pentaglycosyl phosphate repeating unit and PS-II of a hexaglycosyl phosphate repeating unit both occurring on highly virulent strain ribotype 027 (FIG. 1). Said polysaccharides were disclosed as antigenic/-immunogenic and, consequently, of interest for use in vaccine compositions. Ganeshapillai et al. did not disclose or suggest that oligosaccharides derived from the repeating unit of PS-II alone might be a strong antigenic determinant suitable for the development of a vaccine against C. difficile. Furthermore, the exact epitope or length of the surface polysaccharides that are immunogenic or antigenic were not defined.

The present inventors succeeded in the synthesis of a hexasaccharide derived from the repeating unit of the C. difficile polysaccharide PS-II, and its conjugation to the diphteria toxoid Crm₁₉₇. They were further able to produce monoclonal antibodies that specifically recognize the glycan epitope and polysaccharide-specific IgA antibodies were detected in patients diagnosed with C. difficile infections.

Consequently, the above main object of the invention is achieved by providing the oligosaccharide-protein conjugate according to claim 1, the synthetic hexasaccharide according to claim 7 and the vaccine according to claim 9. Related objects are achieved by providing the antibody of claim 14 and the methods of synthesis according to claims 17 and 21. Preferred embodiments and further aspects of the invention are the subject of the dependent claims.

### DESCRIPTION OF THE INVENTION

The present invention provides an oligosaccharide-protein conjugate comprising an oligosaccharide, in particular synthetic oligosaccharide, derived from the repeating unit of the Clostridium difficile glycopolymer PS-II and a protein carrier. More specifically, the oligosaccharide is the hexasaccharide having the following formula I

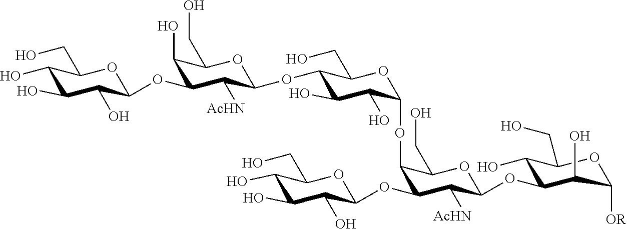

wherein R is a linker or spacer group.

The linker or spacer group R may be any moiety that enables to couple the oligosaccharide to a carrier molecule or to the surface of a microarray. A large variety of such linker groups are known in the art and a suitable linker group can be selected in dependence from the respective carrier molecule or surface group. For example, R may be an aliphatic or aromatic residue comprising a reactive functional group, such as an amino group, preferably a primary amino group, (activated) carboxy group, aldehyde, azide, alkenyl or alkinyl group. In specific embodiments R may comprise a polyether or polyester chain. In particular, R is selected from the group comprising primary alkylamines, alkyl or aralkyl residues with a terminal aldehyde, azide, alkine or alkene group or (activated) carboxy group, and alkylaryl and aryl residues, e.g. phenyl residues, comprising a reactive amine, aldehyde or azide group, or (activated) carboxy group.

In a specific embodiment of the invention, R is (CH₂)ₙNH₂, with n being an integer from 2 to 50, preferably 3 to 20 or 3 to 10, such as 4 to 8.

The carrier may be any carrier molecule known in the art, in particular in the field of vaccine development, e.g. as disclosed in Hecht et al., Curr. Opin. Chem. Biol. 13, 354-359. (2009). More specifically the carrier is a protein carrier selected from the group comprising diphtheria toxoid Crm₁₉7, tetanus toxoid, outer membrane protein (OMP), bovine serum albumin, keyhole limpet hemocyanine.

As demonstrated in the Examples below, the synthetic hexasaccharide derived from the repeating unit of C. difficile PS-II is able to induce an immunogenic and antigenic response in mice and human patients.

Consequently, an aspect of the present invention relates to a vaccine against the pathogen Clostridium difficile comprising at least one of the group consisting of the oligosaccharide-protein conjugate according to claim 1, the hexasaccharide according to claim 7 or a truncated derivative thereof, or a conjugate of the hexasaccharide according to claim 7 or derivative thereof with a non-protein carrier molecule.

The oligosaccharide-protein conjugate or the oligosaccharide, in particular the hexasaccharide, of the invention may be advantageously used for preparing a pharmaceutical composition for the treatment or prevention of a disease caused by a pathogenic strain of Clostridium difficile.

In a related aspect they may be used in a method for the treatment or prevention of a disease caused by the pathogen Clostridium difficile.

A further aspect of the invention relates to an antibody having specifity for an immunogenic determinant derived from or comprising the repeating unit of the Clostridium difficile glycopolymer PS-II. More specifically, the immunogenic determinant comprises or consists of the hexasaccharide of formula I.

In a specific embodiment, said antibody has been raised against the oligosaccharide-protein conjugate according to claim 1.

The antibody may be a polyclonal or monoclonal antibody and monoclonal antibodies can be readily prepared by standard methods of the art (e.g. Köhler and Milstein (1975), Nature, 495-497) as demonstrated in Example 3 below. In one specific embodiment, the monoclonal antibody is the antibody C2805.7 or C2805.21.

The present invention also provides very favourable and efficient methods for synthesizing the hexasaccharide and hexasaccharide-protein conjugate of formula I selectively and in high yields.

Generally, the present method for synthesizing the hexasaccharide comprises assembling the monosaccharide building block 3 of FIG. 1, wherein R is a linker or spacer group, more specifically wherein R is (CH₂)ₙNH₂, with n being an integer from 2 to 50, more specifically from 3 to 10, (in this case 3 being identical with compound 11 in schemes 1 and 2 below) and building block 4

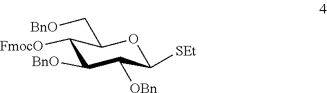

and the disaccharide building blocks 5 or 15

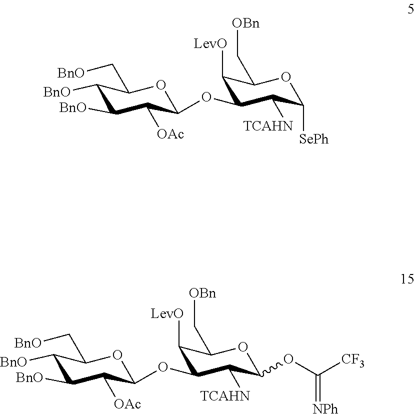

derived from the monosaccharide building blocks 6 and 7

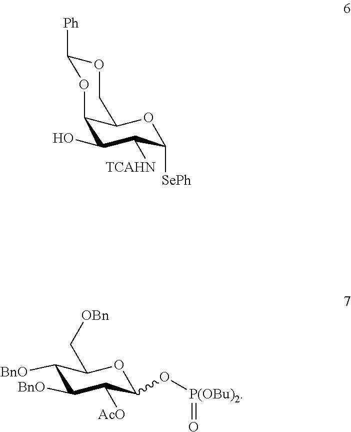

In a more specific embodiment of the invention, in said method building block 3 is obtained by reacting a N-benzyl-N-benzyloxycarbonyl-n-aminoalkanol (with n being an integer of 2-50) with building block 9 of the following formula

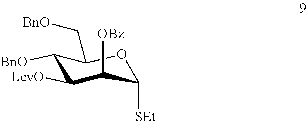

having 2-O-benzoyl and 3-O-levulinoyl protection groups in order to obtain compound 10,

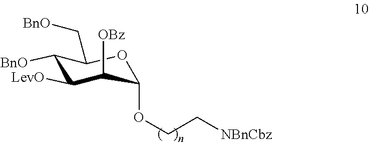

and subsequently selective cleaving of the levulinoyl ester to obtain compound 11 (building block 3 in FIG. 1)

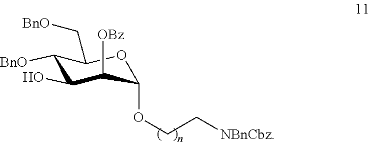

In a preferred embodiment, the method comprises reacting the disaccharide N-phenyl trifluoracetamide 15 (obtained from the phenyl selenide 5)

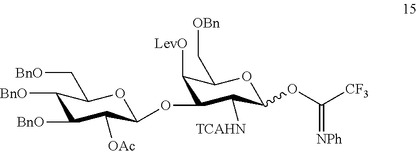

with building block 3, resulting in the trisaccharide 19

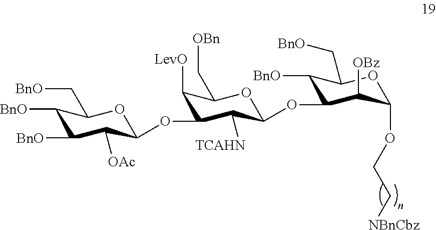

and subsequently cleaving of the levulinoyl ester to obtain compound 20

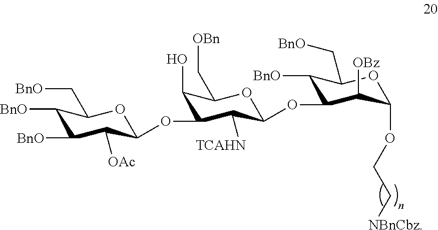

Preferably, the synthesis further comprises reacting compound 20 with thioglycoside 16

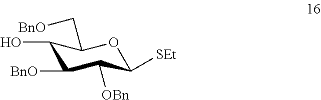

or N-phenyltrifluoracetimidate glycoside 18

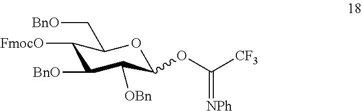

resulting in the tetrasaccharide 21

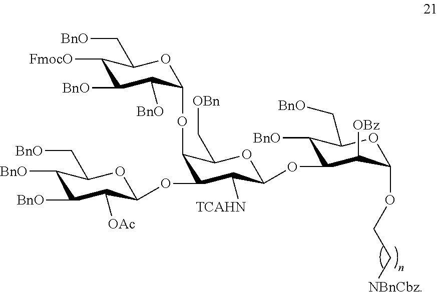

In a further preferred embodiment, the preparation of the building block 5 comprises

i) reacting the monosaccharide building blocks 6 and 7 to obtain the disaccharide compound 12

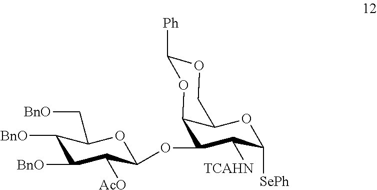

ii) treating compound 12 with triethylsilane and triflic acid to obtain compound 13

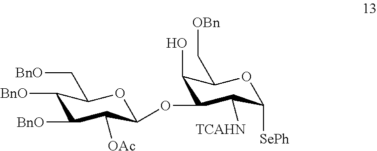

and iii) masking the C4 hydroxyl group of compound 13 as a levulinoyl ester to afford compound 5.

The method for preparing the hexasaccharide-protein conjugate of the present invention typically comprises coupling the hexasaccharide of formula I wherein R is a linker or spacer group, in particular wherein R is (CH₂)ₙNH₂, with n being an integer from 2 to 50, preferably from 3 to 20, with a protein carrier.

More specifically, said method comprises reacting the hexasaccharide of formula I wherein R is (CH₂)ₙNH₂, with n being an integer from 4 to 8, with diethyl squarate to obtain the squarate adduct, in particular compound 24,

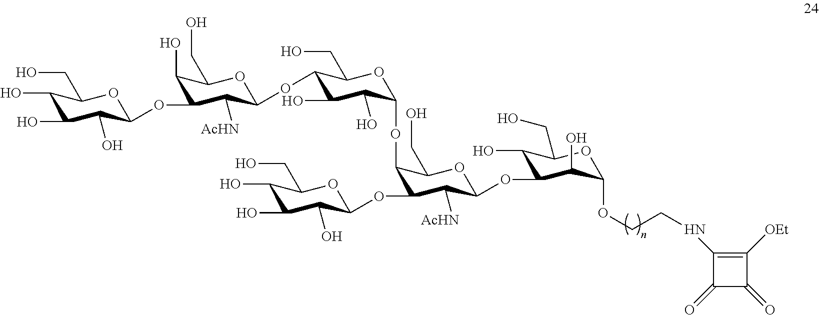

and subsequently coupling the adduct to the protein carrier.

The protein carrier may be any carrier disclosed above and in one specific embodiment the protein carrier is Crm₁₉₇.

In the following, the methods of synthesis according to the invention are outlined in more detail with respect to preferred embodiments but are not limited thereto.

General Oligosaccharide Synthesis

The PS-II repeating units are interconnected via a (1→6) phosphate diester linkage in the natural polysaccharide (FIG. 1). The present inventors developed a very effective method for synthesizing hexasaccharide I that comprises the PS-II repeating unit but differs from the natural hexasaccharide by the group R. In a preferred embodiment, the oligosaccharide was designed to carry a primary amine at the reducing terminus via a spacer to facilitate conjugation to a protein carrier and attachment to microarrays. Based on the retrosynthetic analysis, the hexasaccharide will be assembled from the monosaccharide building blocks 3 and 4, and the disaccharide building block 5 that appears twice in the target structure. Disaccharide 5 will be derived in turn from monosaccharide building blocks 6 and 7.

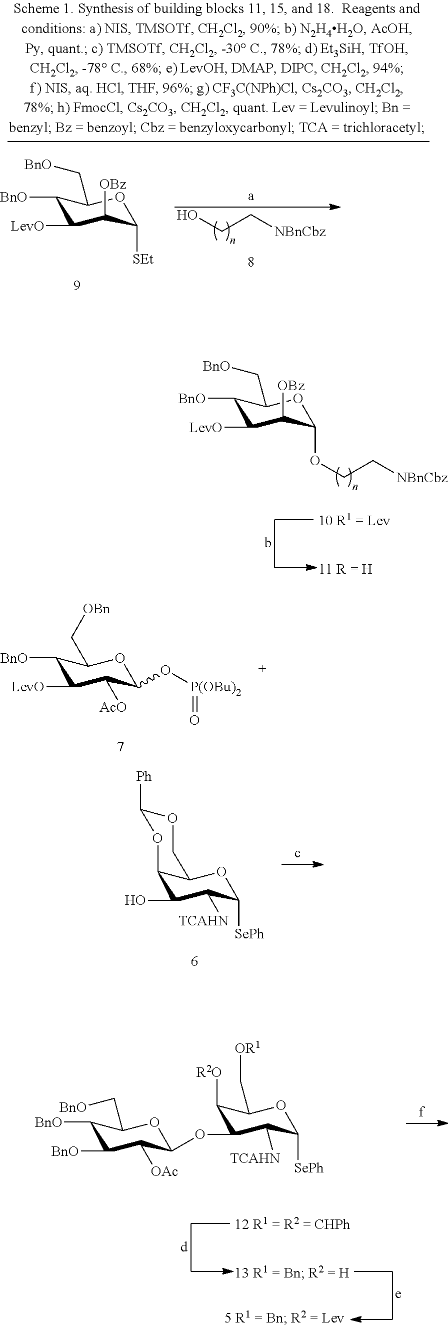

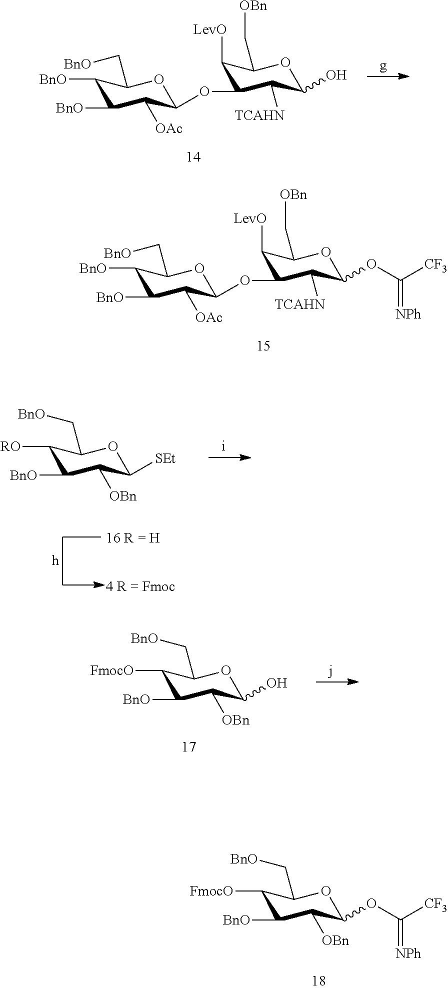

Preparation of reducing terminus commenced with the glycosylation of the protected spacer, e.g. N-benzyl-N-benzyloxycarbonyl-n-aminoalkanol 8 (Scheme 1). The 2-O-benzoyl and 3-O-levulinoyl protection groups on building block 9 were crucial for the success of this glycosylation since the corresponding mannose building block bearing 2-O-acetate and 3-O-fluorenylmethyloxycarbonyl (Fmoc) protecting groups afforded mainly the orthoester product. The levulinoyl ester in mannose glycoside 10 was selectively cleaved using hydrazine monohydrate to reveal the C3 hydroxyl group.

Disaccharide building block 15 resulted from the union of galactosamine 6 with known glucosyl phosphate 7 (Ravidà et al. (2006), Org. Lett. 8, 1815-1818). The selective opening of the benzylidene acetal strongly depended on the reaction conditions. Best results were obtained when disaccharide 12 was treated with triethylsilane and triflic acid at low temperatures. Other methods that rely on triflic acid as Lewis acid or sodium cyanoborohydide as reducing agent furnished inseparable mixtures of the 4-hydroxyl and 6-hydroxyl-regioisomers. The free C4 hydroxyl group in disaccharide 13 was masked as levulinoyl ester to afford glycosylating agent 5. Glycosylation of monosaccharide 11 with phenyl selenide 5 furnished trisaccharide 19 (Depré et al. (1999), Chem. Eur. J. 5, 3326-3340). To improve the coupling yields, the anomeric leaving group of phenyl selenide 5 was converted, via lactol 14, to the corresponding glycosyl N-phenyl trifluoroacetimidate 15.

Hexasaccharide assembly commenced with the glycosylation of nucleophile 11 with disaccharide N-phenyl trifluoroacetimidate 15. The glycosylation yield compared favourably to the yield obtained when phenyl selenide 5 was used. The C2 participating trichloroacetamido group of galactosamine ensured the exclusive formation of the β-linkage. Treatment of trisaccharide 19 with hydrazine monohydrate resulted in cleavage of the levulinoyl ester and furnished 20. Glycosylation of trisaccharide 20 with thioglycoside 16 afforded tetrasaccharide 21. The yield of this glycosylation was improved when N-phenyltrifluoroacetimidate glycoside 18 was employed instead. This glycosylating agent in a mixture of methylene chloride and diethyl ether at −45° C. afforded α-linked glucose containing tetrasaccharide 21 in very high yields. Glucose building block 18 was prepared from known thioglycoside 16 (van Steijn et al. (1992), Carbohydr. Res. 225, 229-245). Treatment of tetrasaccharide 21 with triethylamine resulted in cleavage of the Fmoc carbonate, before the subsequent union of disaccharide glycosylating agent 15 and 22 afforded the desired hexasaccharide 23.

Hexasaccharide 23 was freed from all protecting groups via a three-step procedure. First, the N-trichloroacetyl were transformed into N-acetyl groups by treatment with tributyl stannane and azobisisobutyronitrile (AIBN) in toluene (Scheme 2) (Bélot et al. 2000; Rawat et al. 2008). Subsequent saponification using potassium hydroxide in tetrahydrofurane and methanol was followed by hydrogenation using hydrogen gas and palladium on charcoal. Thereby, the hexasaccharide repeating unit 2 was obtained as shown in Scheme 2 below.

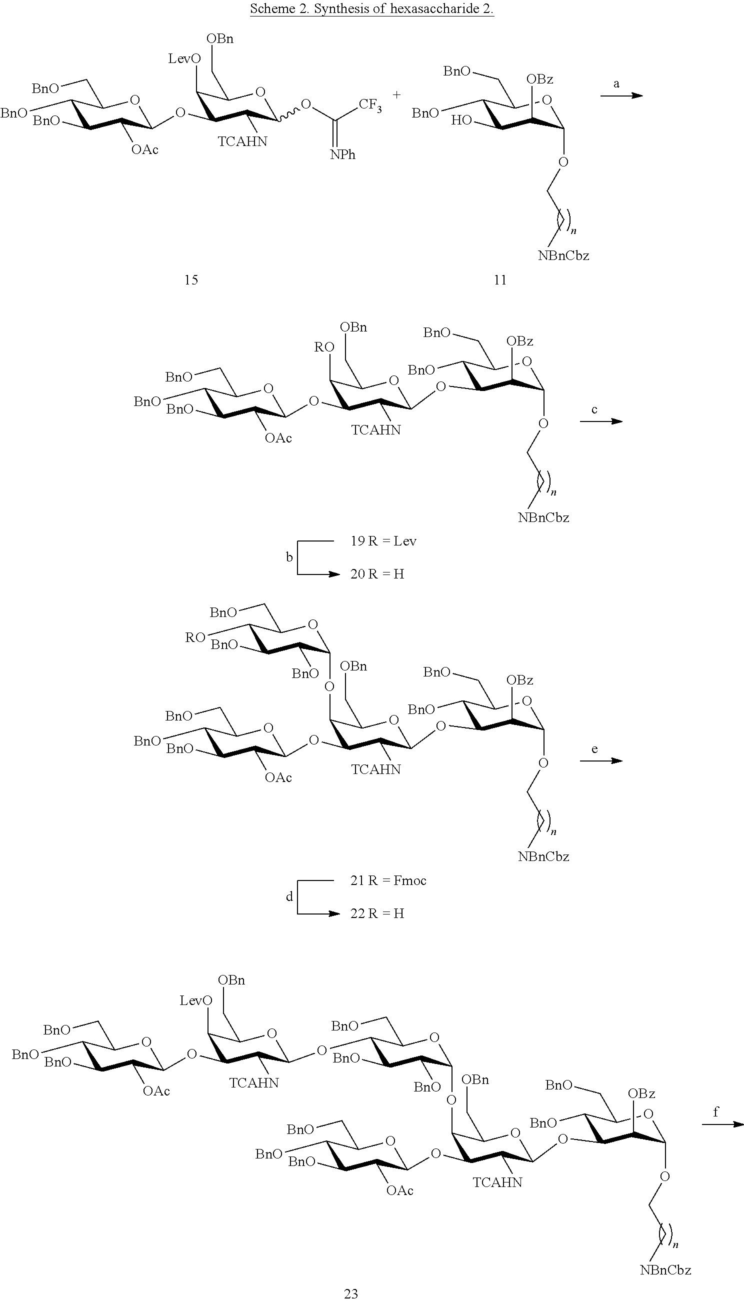

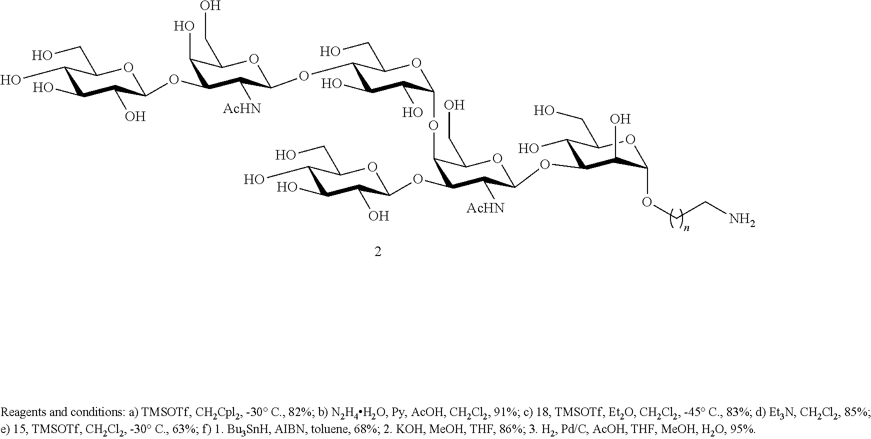

The invention is further illustrating by the following non-limiting Examples and Figures.

### FIGURES

FIG. 1. Retrosynthetic analysis of hexasaccharide repeating unit 2. A) Structure of PS-II polysaccharide found on C. difficile. B) Retrosynthetic analysis of PS-II polysaccharide repeating unit analogue 2.

FIG. 2. Conjugation and Analysis of the Hexasaccharide 2-Crm₁₉₇ Glycoconjugate. A) Hexasaccharide 2a (R═(CH₂)₅NH₂) was reacted with the carrier protein Crm₁₉₇ via squaric acid route to yield a polyvalent neoglycoconjugate. B) SDS-PAGE analysis of the conjugation. Samples were electrophoresed on 12.5% SDS-PAGE gels and stained with Coomassie Blue. B) MALDI-TOF mass spectra of the neoglycoconjugate. Left (blue): Crm₁₉₇ with a m/z peak at 58.5 kDa. Right (red): hexasaccharide 2a-Crm₁₉₇ conjugates with m/z peaks between 59.9 kDa and 67.3 kDa.

FIG. 3. IgA Analysis of Stool Supernatant of Hospitalized Persons. High titers of anti hexasaccharide 2a IgA antibodies were detected in patients 2095 (positive), 2122 (borderline) and 2031 (positive). Low-intensity signals were also detected in patients 2093, 2118, and 2121 (all diagnosed negative).

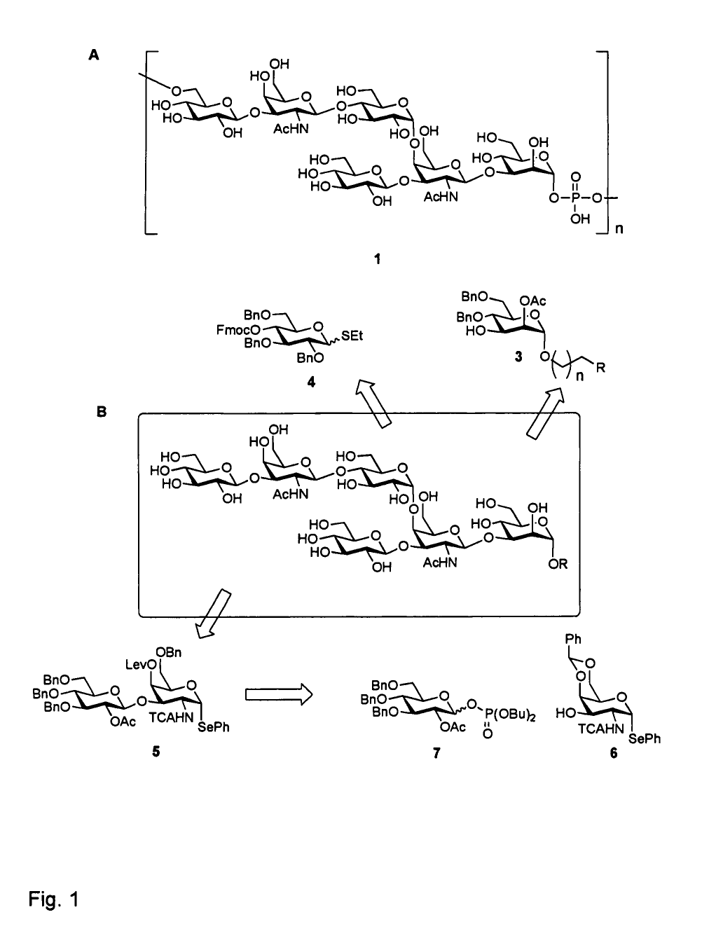

### EXAMPLE 1

### Preparation and Characterization of a Hexasaccharide Based on the Repeating Unit of C. difficile Polysaccharide PS-II

The hexasaccharide was designed to provide, by means of a spacer or linker group, a primary amine at the reducing terminus to facilitate conjugation to a protein carrier and attachment to microarrays. In the following synthesis, the spacer comprises the (CH₂)₅NH₂ group.

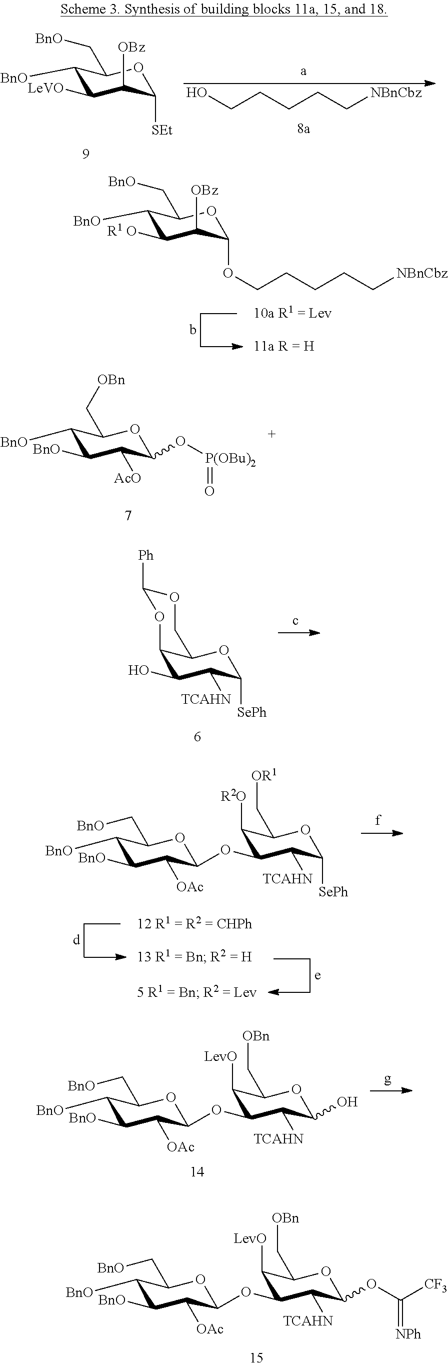

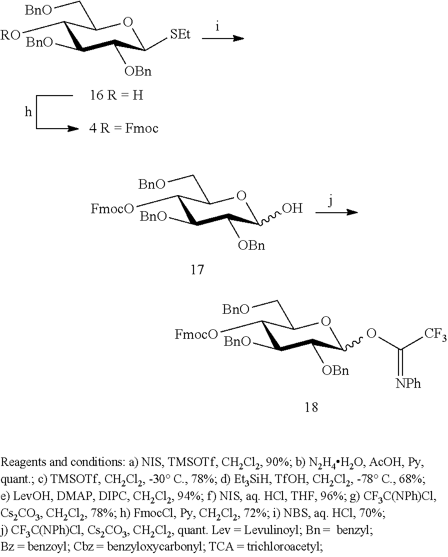

Preparation of reducing terminus commenced with the glycosylation of the protected spacer N-benzyl-N-benzyloxy-carbonyl-5-aminopentanol 8 (Delcros et al. (2002), J. Med. Chem. 45, 5098-5111.) (see Scheme 3). The levulinoyl ester in mannose glycoside 10a was selectively cleaved using hydrazine monohydrate to reveal the C3 hydroxyl group.

Disaccharide building block 15 resulted from the union of galactosamine 6 with glucosyl phosphate 7. The selective opening of the benzylidene acetal strongly depended on the reaction conditions. Best results were obtained when disaccharide 12 was treated with triethylsilane and triflic acid at −78° C. Other methods that rely on triflic acid as Lewis acid or sodium cyanoborohydide as reducing agent furnished inseparable mixtures of the 4-hydroxyl and 6-hydroxyl-regioisomers. The free C4 hydroxyl group in disaccharide 13 was masked as levulinoyl ester to afford glycosylating agent 5. Glycosylation of monosaccharide 11a with phenyl selenide 5 furnished trisaccharide 19 in up to 61% yield. To improve the coupling yields, the anomeric leaving group of phenyl selenide 5 was converted, via lactol 14, to the corresponding glycosyl N-phenyl trifluoroacetimidate 15.

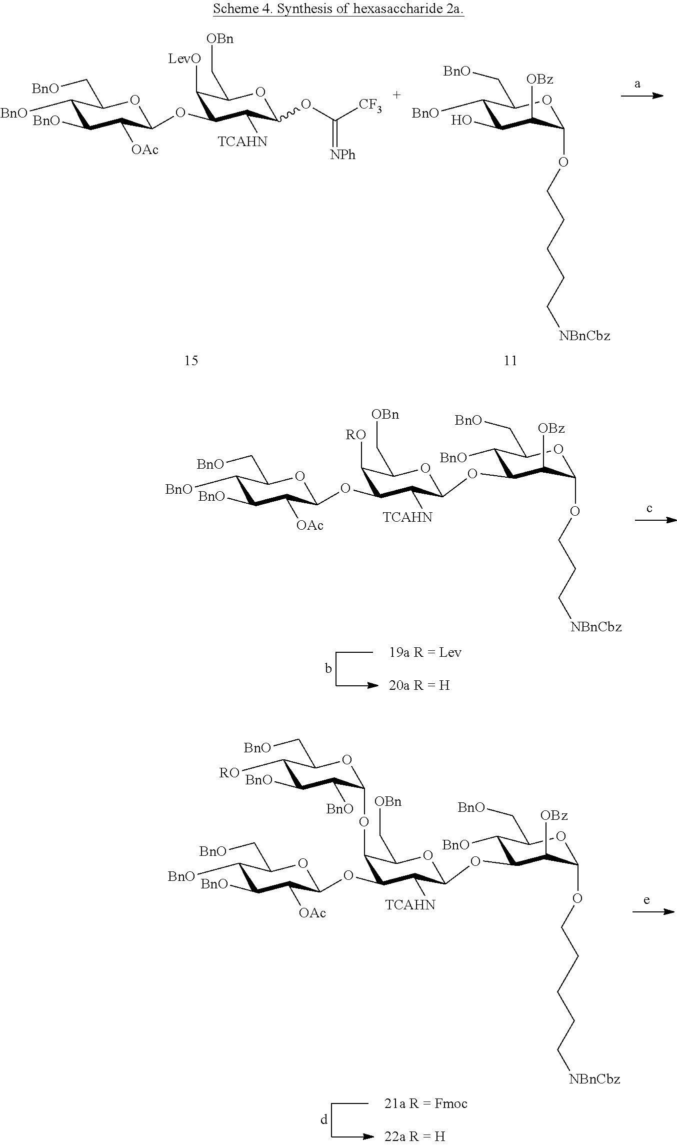

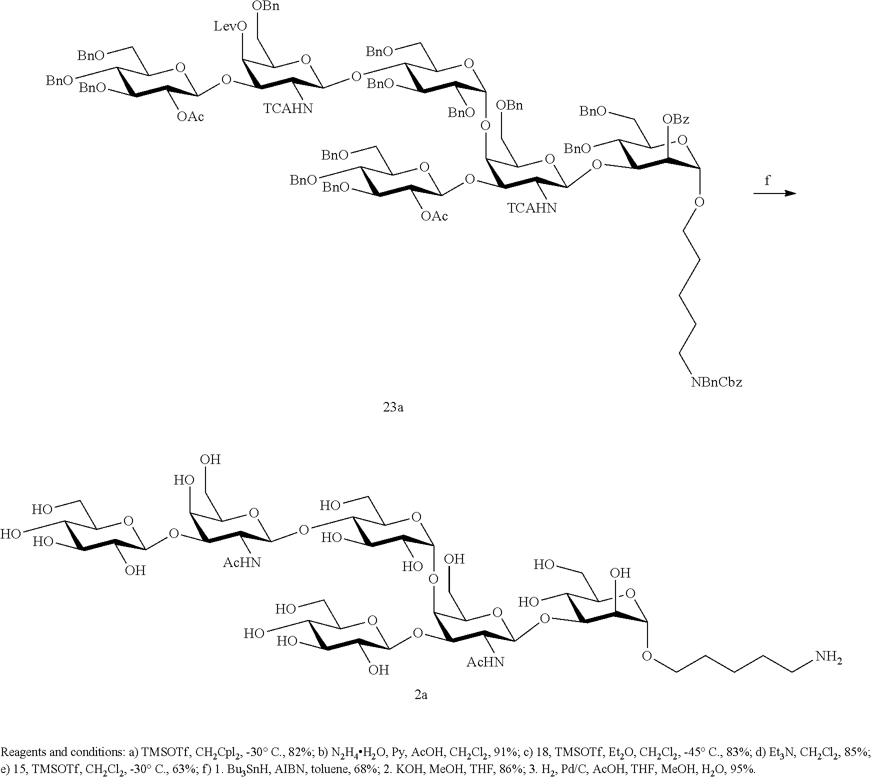

Hexasaccharide assembly commenced with the glycosylation of nucleophile 11a with disaccharide N-phenyl trifluoroacetimidate 15. The glycosylation yield of 82% compared favourably to the 61% obtained when phenyl selenide 5 was used. The C2 participating trichloroacetamido group of galactosamine ensured the exclusive formation of the β-linkage. Treatment of trisaccharide 19a with hydrazine monohydrate resulted in cleavage of the levulinoyl ester and furnished 20a. Glycosylation of trisaccharide 20a with thioglycoside 16 afforded tetrasaccharide 21a in 55% yield. The yield of this glycosylation was improved when N-phenyltrifluoroacetimidate glycoside 18 was employed instead. This glycosylating agent in a mixture of methylene chloride and diethyl ether at −45° C. afforded α-linked glucose containing tetrasaccharide 21a in 83% yield. Glucose building block 18 was prepared from known thioglycoside 16. Treatment of tetrasaccharide 21a with triethylamine resulted in cleavage of the Fmoc carbonate, before the subsequent union of disaccharide glycosylating agent 15 and 22a afforded the desired hexasaccharide 23a.

Hexasaccharide 23a was freed from all protecting groups via a three-step procedure. First, the N-trichloroacetyl groups were transformed into N-acetyl groups by treatment with tributyl stannane and azobisisobutyronitrile (AIBN) in toluene at 90° C. (Scheme 2). Subsequent saponification using potassium hydroxide in tetrahydrofurane and methanol was followed by hydrogenation using hydrogen gas and palladium on charcoal. Thereby, the hexasaccharide 2a was obtained.

Key regions in the NMR spectra of synthetic hexasaccharide 2a and isolated polysaccharide 1 differ slightly, as expected. The NMR signals of the α-mannoses are different since the mannose of the synthetic hexasaccharide is equipped with an aliphatic spacer at the reducing end while the repeating units in the natural polysaccharide are connected via phosphate diester linkages. Aside from these expected differences, the spectra confirm the structural assignment.

### EXAMPLE 2

### Preparation and Characterization of an Hexasaccharide-Protein Conjugate

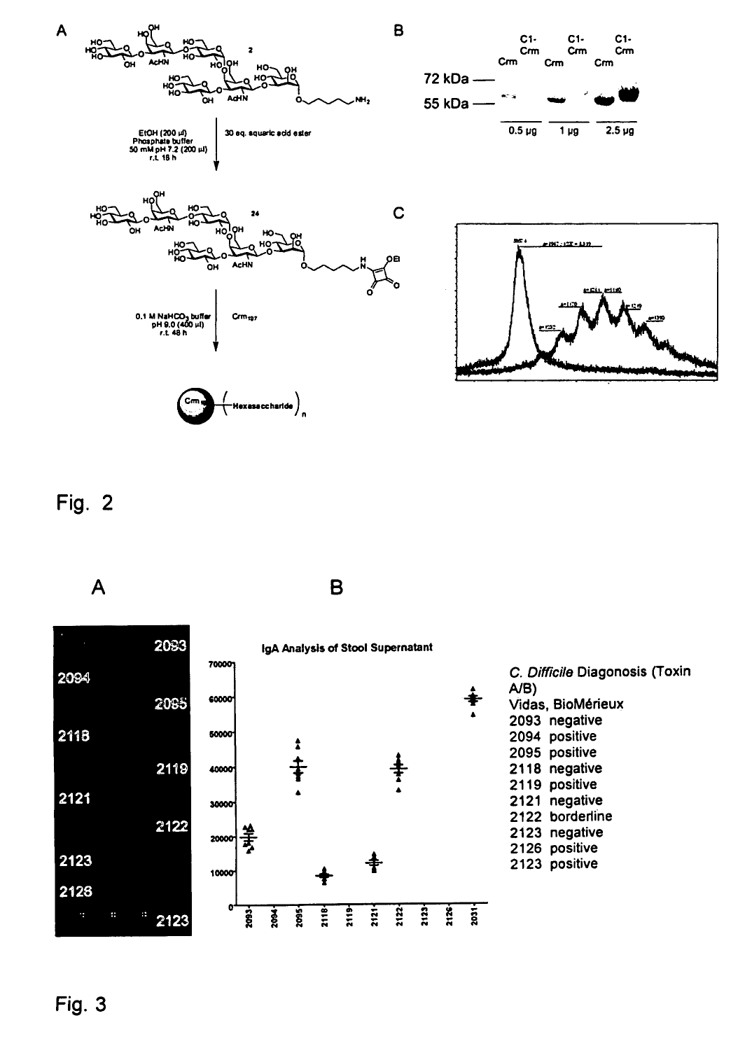

Polysaccharide vaccines provoke exclusively a T-cell independent immune response and do not induce an immunoglobulin class switch. The synthetic repeating unit 2, in particular 2a, of the Clostridium difficile glycopolymer PS-II was conjugated to the protein carrier Crm₁₉₇. The tetoxified diphtheria toxoid Crm₁₉₇ was chosen as a carrier since it is an approved constituent of licensed vaccines (Barocchi et al. (2007), Vaccine 25, 2963-73). A method based on the selective reaction of the primary amine with squaric acid diester (Tietze et al. (1991), Bioconjugate Chem. 2, 148-153) was selected from the multitude of methods for conjugation of carbohydrates to proteins (Kuberan et al. (2000), Curr. Org. Chem. 4, 653-677; Hecht et al. (2009). First, the amine group of the spacer moiety in hexasaccharide (2a) was reacted with one of the ester groups of 3,4-di-ethoxy-3-cyclobutene-1,2-dione in pH 7.2 phosphate buffer to form the corresponding monoamine 24a that was purified by reverse phase HPLC chromatography (FIG. 2A). The remaining ester group of monoamide 24a was subsequently coupled with the ε-amino groups of lysine on the diphtheria toxoid Crm₁₉₇ in bicarbonate buffer at pH 9.0 to afford the neoglycoconjugate.

Successful conjugation was confirmed by SDS-PAGE (FIG. 2B) and the oligosaccharide/Crm₁₉₇ ratio was determined by MALDI-TOF (FIG. 2C). The mass analysis of Crm₁₉₇ yielded a m/z ion at 58.6 kDa. The mass spectrum of the neoglycoconjugate revealed mass peaks between 59.9 kDa and 67.3 kDa corresponding to mono- to heptavalent glycoconjugates. On average of four hexasaccharides 2 (2a) were loaded on the diphtheria toxoid.

Conjugation. Diethyl squarate (7.3 μL, 51 μmol) was added to a solution of hexasaccharide Cl (2 mg, 1.7 μmol) in EtOH (0.2 mL) and phosphate buffer (0.2 mL, 50 mM, pH 7.2) and stirred for 18 h at room temperature. Most ethanol was removed by a stream of N₂. The mixture was purified using a HPLC superdex size exclusion column (95:5 H₂O, EtOH) to afford a colorless solid. ESI-HRMS: m/z calcd for C₅₁H₈₄N₃O₃₄ [M+Na]⁺ 1304.4756, obsd 1304.4774. A solution of the squarate adduct (0.7 mg, 545.8 nmol) and the diphtheria toxoid Crm₁₉₇ (Calbiochem, 0.7 mg, 11.1 nmol) in NaHCO₃ buffer solution (0.4 mL, 0.1 M, pH 9) was shaken for 48 h at room temperature. The resulting mixture was purified by ultrafiltration (30 K, Amicon, Millipore) with PBS. The protein concentration was determined by Bradford analysis (Biorad).

SDS-PAGE. Hexasaccharide 2a-Crm₁₉₇ glycoconjugate and unconjugated Crm₁₉₇ were dissolved in Lämmli buffer (0.125 M Tris, 20% (v/v) glycerol, 4% (w/v) SDS, 5% (v/v) β-mercaptoethanol, bromphenol, pH 6.8) and boiled for 5 min. Samples were run in 12.5% polyacrylamide gel and stained with 0.025% (w/v) Coomassie Brilliant blue R-250 in an aqueous solution containing 40%(v/v) methanol and 7% (v/v) acetic acid.

MALDI-TOF Mass Spectrometry. Conjugation was confirmed by matrix-assisted laser desorption ionization-time of flight MS (MALDI-TOF-MS) using an Ultraflex-II TOF/TOF instrument (Bruker, Daltonics, Bremen, Germany) equipped with a 200 Hz solid-state Smart beam™ laser. The mass spectrometer was operated in the positive linear mode. MS spectra were acquired over an m/z range 40,000-80,000 and data was analyzed using FlexAnalysis® software provided with the instrument. The samples were lyophilized from 25 mM NH₄HCO₃ (pH 7.8). Sinapinic acid was used as the matrix and samples were spotted using the dried droplet technique.

### EXAMPLE 3

### Immunization and Monoclonal Antibodies

In order to test the immunogenicity of the conjugate, two female C57BL/6 mice were immunized with the neoglycoconjugate. Mice were injected three times subcutaneously with 15 μg of the glycoconjugate in two-week intervals. The anti-hexasaccharide 2 (2a) antibody titers were monitored by glycan microarray analysis. Microarrays were designed for high-throughput analysis, such that 64 samples can be analyzed on one array with each well displaying hexasaccharide 2 (2a) and seven control sugars in quadruplicates. Two immunized mice produced IgG antibodies that bound specifically to hexasaccharide 2 (2a) to demonstrate that 2a is immuno-reactive. Affinity maturation of the anti-hexasaccharide 2a IgG antibodies was observed with mouse 2805. Polyclonal IgG antibodies were raised against hexasaccharide 2a to demonstrate the immunogenicity of the C. difficile cell surface glycopolymer PS-II.

To generate monoclonal antibodies, splenocytes of the immunized mice were fused to myeloma cells by the traditional hybridoma technique (Köhler and Milstein, Nature (1975), 256, 495-497). The individual hybridoma clones were screened to identify clones that produce anti-hexasaccharide 2a antibodies. Three hybridoma clones that secrete specific antibodies were obtainedAll three hybridoma clones were derived from mouse 2805, the animal whose IgG antibodies had undergone affinity maturation during immunization. While the monoclonal antibodies C2805.7 and C2805.21 bound exclusively to hexasaccharide 2a antibody C2805.25 also interacted with glucose on the array.

Immunizations. Two female C57BL/6 mice were immunized s.c. with 15 μg hexasaccharide 2a-Crm₁₉₇ in Freund's complete adjuvants. The mice were boosted twice with 15 μg hexasaccharide 2a-Crm₁₉₇ conjugate in Freund's incomplete adjuvants in two-week intervals. After each injection blood was collected and serum titers (IgG) were analyzed using microarrays. Prior to being sacrificed, mice received 10 μg hexasaccharide 2a-Crm₁₉₇ in PBS i.p. on three consecutive days.

Preparation of Clostridium Microarrays. Eight oligosaccharides bearing an amine linker were immobilized on NHS-activated slides. Besides hexasaccharide 2a, mannose, glucose, galactose, fucose, N-Acteylglucosamine, lactose and a β-galactoside³³⁷ were printed in 0.5 mM concentration onto the slides. Each spot was printed in quadruplicate using a piezoelectric spotting device (S11, Scienion, Berlin, Germany). Slides were incubated in a humid chamber to complete reaction for 24 h and stored in a dessicator until usage.

Microarray Binding Assays. A FlexWell 64 (Grace Bio-Labs, Bend, USA) grid was applied to the slides. The resulting 64 wells were used for 64 individual experiments. The slide was blocked with 2.5% (w/v) BSA and 0.05% (v/v) Tween20 in PBS for 1 h at room temperature. Blocked slides were washed with PBS and incubated with 5% (v/v) serum in PBS or hybridoma culture supernatant for 1 h at room temperature. Slides were washed with PBS and incubated with 10 μg/mL Alexa Fluor 594 goat anti-mouse IgG and Alexa Flour 594 goat anti-mouse IgM (both Invitrogen) secondary antibody solutions in PBS with 1% (w/v) BSA. Slides were washed with PBS and centrifuged to dryness. Slides were scanned using a GenePix 4300A scanner (Bucher biotec, Basel, Switzerland) and evaluated using the GenePix Pro7 software (Bucher biotec, Basel, Switzerland).

Monoclonal Antibody Purification. Supernatant of the hybridoma clones was filtered through a 0.2 μm filter. The supernatant was mixed 1:1 with binding buffer (0.1 M NaP, 0.15 M NaCl pH 7.4) and loaded onto a Midi Protein G spin column (Proteus, Oxford, UK). The spin column was washed twice with binding buffer. Subsequently, the IgG was eluted with elution buffer (0.2 M glycane/HCl pH 2.5) and immediately neutralized with 1 M Tris/HCl pH 9. The eluted antibody solution was purified by ultrafiltration (100 K, Amicon, Millipore) with PBS containing 0.01% (w/v) sodium azide. Protein stabilizing cocktail (Pierce, Rockford, USA) was added to the concentrated antibody solution and the protein concentration was determined by Bradford analysis (Biorad).

### EXAMPLE 4

### Detection of Specific IgA Antibodies in Infected Hospital Patients

Given the immunogenicity of PS-II in mice, it should be assessed whether CDAD patients produce antibodies against the native glycopolymer. To this end, stool supernatants of ten hospitalized patients with and without C. difficile infection as confirmed by the VIDAS® immunoassay (bioMérieux) that detects toxin A and B were analyzed. Stool supernatant rather than serum was chosen because the contact site of the immune system with the cell surface glycopolymer is the intestinal mucosa. Glycan arrays were incubated with the stool supernatant and bound IgA antibodies were visualized. Three persons had high titers of anti-hexasaccharide 2a IgA antibodies in their stool (FIG. 3). Of these three patients, two had been diagnosed as C. difficile toxin A/B-positive while the third patient had a borderline VIDAS® test. Low amounts of anti-hexasaccharide 2a recognizing IgA antibodies were also detected in patients 2093, 2118 and 2121, which had not been diagnosed C. difficile toxin positive. A possible explanation is colonization with a non-toxigenic C. difficile strain or previous contact with the bacterium.

Analysis of Stool Supernatant. A FlexWell 64 (Grace Bio-Labs, Bend, USA) grid was applied to the slides. The wells were blocked with 2.5% (w/v) BSA and 0.05% (v/v) Tween20 in PBS for 1 h at room temperature. Blocked slides were washed with PBS and incubated with 20 μL stool supernatant of ten hospitalized persons (Charité, Berlin) for 1 h at room temperature. Slides were washed with PBS and incubated with 10 μg/mL goat anti-human IgA FITC Conjugate (Invitrogen) secondary antibody solutions in PBS with 1% (w/v) BSA. Slides were washed with PBS and centrifuged to dryness. Slides were scanned using a GenePix 4300A scanner (Bucher biotec, Basel, Switzerland) and evaluated using the GenePix Pro7 software (Bucher biotec, Basel, Switzerland).

Summarizing, here is reported the first synthesis of C. difficile PS-II hexasaccharide repeating unit that confirms the structural assignment based on isolated material. The repeating unit was assembled from four monosaccharide building blocks using an efficient and convergent approach. A neoglycoconjugate comprising the hexasaccharide repeating unit and the immunogenic carrier protein Crm₁₉₇ was obtained. The outcome of the conjugation process was monitored by MALDI-TOF mass spectrometry and SDS-PAGE. Mice were immunized with the neoglycoconjugate and IgG antibody production against hexasaccharide 2a was monitored by glycan microarray analysis. Two animals produced specific antibodies, one of which showed a gradual increase of the antibody's affinity over the immunization period.

High-throughput carbohydrate microarray analysis served as a fast method to detect antibodies in murine sera, hybridoma supernatant and human excrement. Active ester conjugation chemistry allowed for facile immobilization of the amine-terminated synthetic hexasaccharide antigen to glass slides. In addition to hexasaccharide 2a, seven control carbohydrates were printed onto the microarray slides that were stable for more than one year. Carbohydrate microarray analysis gave a detailed picture of the presence of antibodies, antibody affinity and concentration, as well as cross-reactivity.

Using the microarrays, specific anti-glycopolymer IgA antibodies were detected in stool supernatants of hospital patients. Two patients with significantly increased C. difficile toxin A and B levels and one patient with a borderline test displayed high amounts or highly specific anti-hexasaccharide 2a IgA antibodies in their excrement. These observations suggest that native glycopolymer PS-II is an antigenic determinant upon human infection of with C. difficile. Antibodies in stool are subject to different dilutions depending on the amount of daily elimination, therefore small variations in the concentrations of the individual samples are likely. The three false negative results may be explained by the fact that these individuals were infected with C. difficile strains that do not express PS-II. For the strains prevalent in European hospitals (Zaiss NH, Emerg. Infect. Disease 2010, 16(4).675), the expression of PS-II was confirmed for ribotype 027 and other strains. Since ribotyping is not performed routinely in European hospitals, the genetic background of the pathogens responsible for the infections analysed in this study remains elusive. The low binding signal recorded for three samples of patients without diagnosed C. difficile infection can be accounted for by latent or previous infections with bacteria of the clostridium type carrying PS-II.

In conclusion, the synthesis of the hexasaccharide repeating unit of a C. difficile cell surface polysaccharide gave access to chemically defined and structurally homogeneous material equipped with a primary amine handle. This handle allowed for conjugation of the synthetic repeating unit to the immunogenic carrier protein Crm₁₉₇ and to glass surfaces to produce microarrays. The neoglycoconjugate was immunogenic in mice and produced murine monoclonal antibodies that interact specifically with the glycan repeating unit. The antibody binding specificities were determined by microarray analysis. Furthermore, microarrays were used to detect IgA antibodies in the stool supernatant of infected hospital patients. The presence of antibodies in infected patients suggests a pivotal role of the PS-II polysaccharide in the pathogenesis of C. difficile associated diseases (CDAD). Thus, both the natural polysaccharide and the synthetic sub-structure represent C. difficile vaccine candidates.

1. An oligosaccharide-protein conjugate comprising an oligosaccharide representing part of a repeating unit of the Clostridium difficile glycopolymer PS-II and a protein carrier.

2. The oligosaccharide-protein conjugate according to claim 1, wherein the oligosaccharide is a hexasaccharide having the following formula I or a truncated fragment thereof 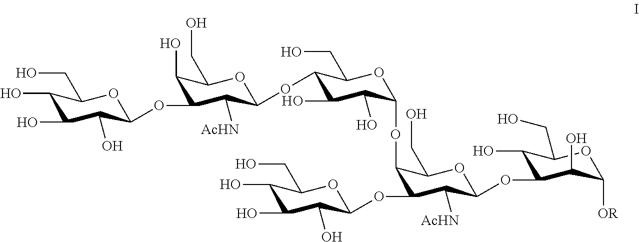
wherein R is a linker or spacer group.

3. The oligosaccharide-protein conjugate according to claim 2, wherein R is an aliphatic or aromatic residue comprising a reactive functional group.

4. The oligosaccharide-protein conjugate according to claim 3, wherein R is (CH₂)ₙNH₂, with n being an integer from 2 to 50.

5. The oligosaccharide-protein conjugate according to claim 1, wherein the protein carrier is selected from the group consisting of diphtheria toxoid Crm₁₉₇, tetanus toxoid, outer membrane protein (OMP), bovine serum albumin, and keyhole limpet hemocyanine

6. The oligosaccharide-protein conjugate according to claim 2, wherein the protein carrier is diphtheria toxoid Crm₁₉₇.

7. A hexasaccharide having the following formula I 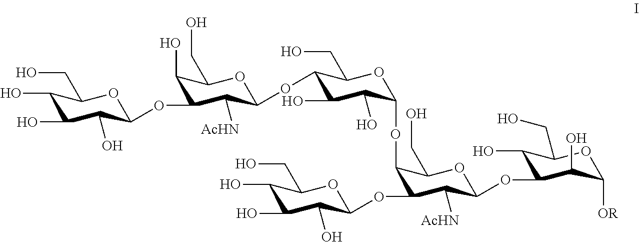
wherein R is a linker or spacer group, with the proviso that R is not a phosphate group.

8. The hexasaccharide according to claim 7, wherein R is (CH₂)ₙNH₂, with n being an integer from 2 to 50.

9. A vaccine against the pathogen Clostridium difficile comprising at least one member selected from the group consisting of:
(a) the oligosaccharide-protein conjugate according to claim 1,
(b) the hexasaccharide having the following formula I 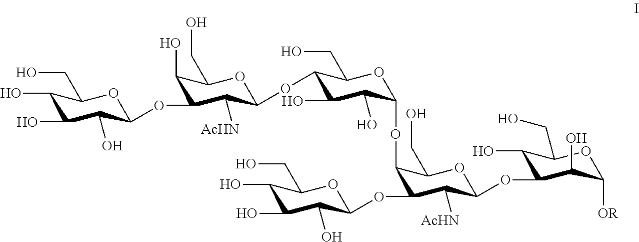
wherein R is a linker or spacer group, with the proviso that R is not a phosphate group, or
(c) a conjugate of the hexasaccharide with a non-protein carrier molecule.

10. The hexasaccharide according to claim 7, which is effective to treat or prevent a disease caused by the pathogen Clostridium difficile.

11. The oligosaccharide-protein conjugate according to claim 1, which is effective to treat or prevent a disease caused the pathogen Clostridium difficile.

12. An antibody having specificity for an immunogenic determinant derived from or comprising repeating unit of the Clostridium difficile glycopolymer PS-II.

13. The antibody according to claim 12, wherein the immunogenic determinant comprises a hexasaccharide having the following formula I 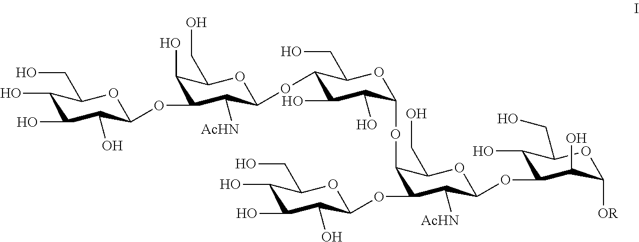
wherein R is a liner or spacer group, with the proviso that R is not a phosphate group, or truncated derivative thereof.

14. The antibody according to claim 13 which has been raised against an oligosaccharide-protein conjugate comprising an oligosaccharide representing part of a repeating unit of the Clostridium difficile glycopolymer PS-II and a protein carrier.

15. The antibody according to claim 13 which is a polyclonal or monoclonal antibody.

16. The monoclonal antibody of claim 15 which is the antibody C2805.7 or C2805.21.

17. A method for preparing the hexasaccharide of claim 7 which comprises assembling compound 11 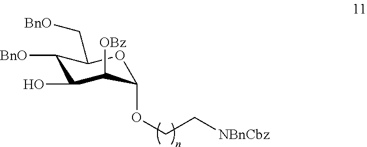
and compound 4 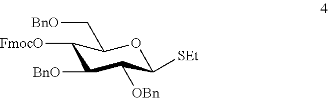
and disaccharide building blocks 5 or 15 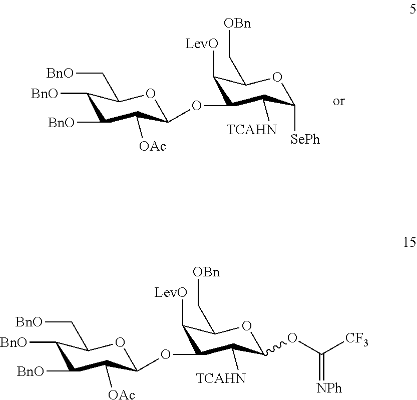
derived from monosaccharide building blocks

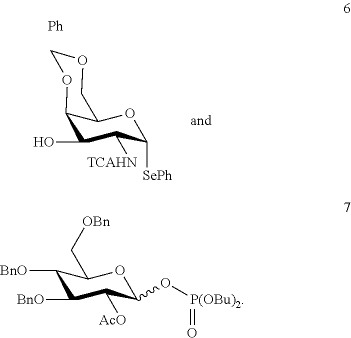

18. The method according to claim 17, wherein building block 3 is obtained by reacting a N-benzyl-N-benzyloxycarbonyl-n-aminoalkanol (with n being an integer of 2-50) with building block 9 of the following formula 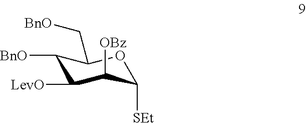
having 2-O-benzoyl and 3-O-levulinoyl protection groups in order to obtain compound 10, 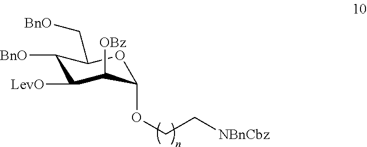
and subsequently selective cleaving of a levulinoyl ester to obtain building block 3 (compound 11 below)

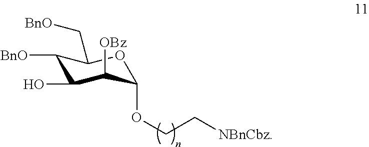

19. The method of claim 17 comprising reacting the di-saccharide N-phenyl trifluoracetamide 15 (obtained from the disaccharide building block 5) 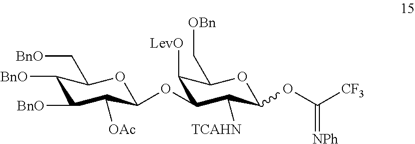
with building block 3, resulting in the trisaccharide 19 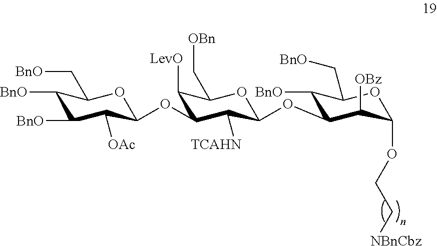
and subsequently cleaving of a levulinoyl ester to obtain compound 20

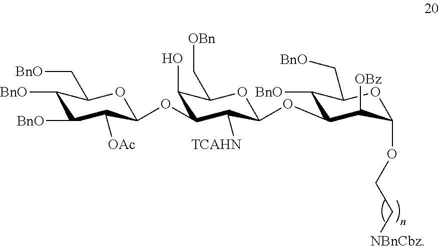

20. The method of claim 19, further comprising reacting compound 20 with thioglycoside 16 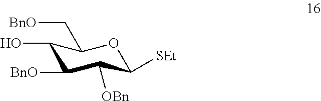
or N-phenyltrifluoracetimidate glycoside 18 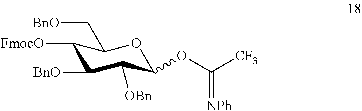
resulting in the tetrasaccharide 21

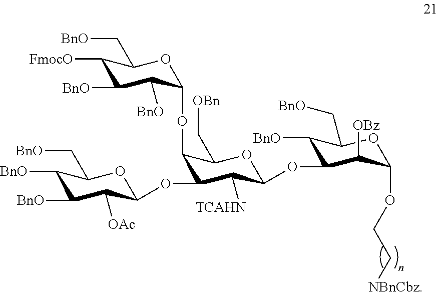

21. The method of claim 17, wherein the building block 5 is prepared by a process comprising:
i) reacting the monosaccharide building blocks 6 and 7 to obtain the disaccharide compound 12 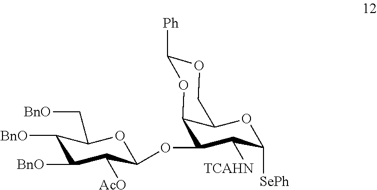
ii) treating compound 12 with triethylsilane and triflic acid to obtain compound 13 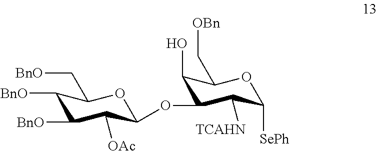
and iii) masking the C4 hydroxyl group of compound 13 as a levulinoyl ester to afford compound 5.

22. A method for preparing the oligosaccharide-protein conjugate according to claim 2 comprising coupling with a protein carrier a hexasaccharide having the following formula I 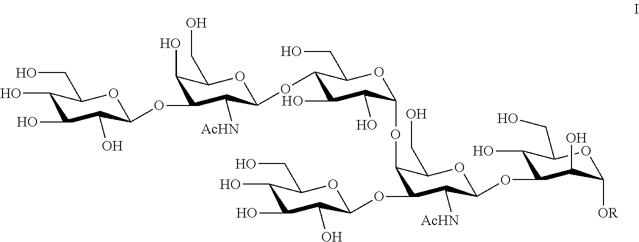
wherein R is a linker or spacer group, with the proviso that R is not a phosphate group.

23. The method according to claim 22, wherein R is (CH₂)ₙNH₂, with n being an integer from 2 to 50 and the method further comprises reacting the hexasaccharide with diethyl squarate to obtain the squarate adduct compound 24, 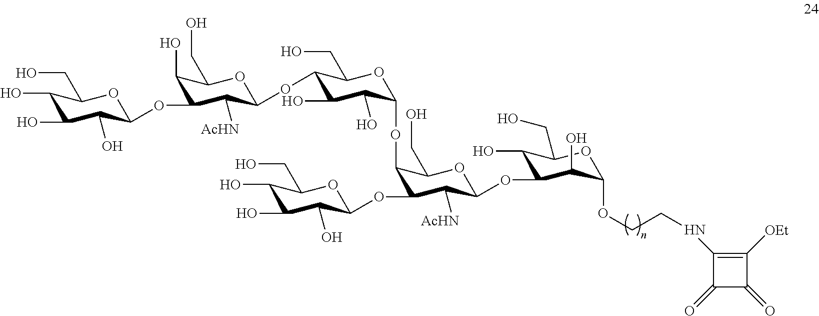
and subsequently coupling the adduct to the protein carrier.

24. The method according to claim 23 wherein the protein carrier is Crm₁₉₇, tetanus toxoid, outer membrane protein (OMP), bovine serum albumin, or keyhole limpet hemocyanine.
